(19) United States
(12) Patent Application Publication
(10) Pub. No.: US 2026/0263321 A1
(43) Pub. Date: Sep. 10, 2026

(54) THERMO-MASSAGE DEVICE HAVING UV LED

(71) Applicant: CERAGEM CO., LTD., Cheonan-si, Chungcheongnam-do (KR)

(72) Inventors: Hyun Jin CHOI, Icheon-si, Gyeonggi-do (KR); Geun Yong JUNG, Yongin-si, Gyeonggi-do (KR); Chang Min YOO, Cheonan-si, Chungcheongnam-do (KR); Han Soo Kim, Dongjak-gu, Seoul (KR)

(73) Assignee: CERAGEM CO., LTD., Cheonan-si, Chungcheongnam-do (KR)

(21) Appl. No.: 19/162,901

(22) PCT Filed: Mar. 6, 2024
(86) PCT No.: PCT/KR2024/002885

§ 371 Date: Sep. 5, 2025

(30) Foreign Application Priority Data
Mar. 7, 2023 (KR) 10-2023-0029902

(51) Int. Cl. A61H 37/00

(52) U.S. Cl. CPC A61H 37/00; A61H 2201/0188; A61H 2201/0207; A61H 2201/10; A61H 2201/5058

(57) ABSTRACT

The present invention relates to a massage device and, more specifically, to a thermo-massage device having a UV LED. The thermo-massage device having a UV LED, according to one aspect of the present invention, comprises: a main body unit having a massage module for thermo-massaging the body of a user, and a first support surface for supporting a portion of the body of the user; an auxiliary unit which has a second support surface for supporting the remaining portion of the body of the user, and which is movably provided so that the first support surface and the second support surface are arranged in a line in the longitudinal direction when the massage module is in use, and the second support surface is arranged above the first support surface in the height direction if the massage module is not in use; a sterilization unit having a UV LED so as to sterilize the first support surface and/or the second support surface; and a control unit for controlling operation of the massage module and the sterilization unit.

### CROSS-REFERENCE TO RELATED PATENT APPLICATIONS

This application is a National Stage filing of International Application No. PCT/KR2024/002885, filed on Mar. 6, 2024, which claims the benefit of and priority to South Korea Patent Application No. 10-2023-0029902, filed on Mar. 7, 2023, the entire disclosures of which are hereby incorporated by reference herein.

### TECHNICAL FIELD

The present invention relates to a massage device and, more specifically, to a thermo-massage device having a UV LED.

### BACKGROUND

In the related art, spinal thermo-massage devices have been widely used which move along a part of the body to apply heat stimulation to an area of pain in order to relieve acute or chronic pain occurring in muscles and nerve tissues of the spinal region caused by continuing to work in an improper posture for a long time or by habitual adoption of such posture, to improve blood circulation in the body, or to relieve momentary muscle stiffness, thereby improving blood flow.

Recently, as the sharing economy has become more active, cases in which a single massage device is used by multiple users have increased. In addition, as massage devices have gained popularity, the number of cases in which users try out displayed massage devices before purchasing them has also increased.

However, when a massage device used by multiple users is not sterilized, infectious diseases may be transmitted between people, and odors or the like left by a previous user may cause discomfort to another user, which becomes problematic.

To address this, massage devices with an added sterilization function have been commercialized; however, such a sterilization function is limited to massage devices of a seated type in which the user sits.

Therefore, there is a need for the development of a technology capable of solving these problems.

### SUMMARY

The present invention is to solve the above problems, and the present invention is directed to providing a thermo-massage device equipped with a UV LED having a sterilization function, which is applicable even to a recumbent-type thermo-massage device used by a user in a lying position.

The problems of the present invention are not limited to those mentioned above, and other problems not mentioned will be clearly understood by those of ordinary skill in the art from the following description.

The thermo-massage device having a UV LED, according to an aspect of the present invention, comprises a main body unit having a massage module configured to thermo-massage a body of a user and a first support surface for supporting a portion of the body of the user; an auxiliary unit which has a second support surface for supporting a remaining portion of the body of the user, and which is movably provided so that, when the massage module is in use, the first support surface and the second support surface are arranged in a line in a longitudinal direction, and, when the massage module is not in use, the second support surface is arranged above the first support surface in a height direction; a sterilization unit having a UV LED configured to sterilize at least one of the first support surface and the second support surface; and a control unit configured to control operations of the massage module and the sterilization unit.

In this case, the thermo-massage device may further include a detection unit configured to detect whether a user is present and whether the second support surface is arranged above the first support surface in the height direction, and the control unit may be configured to control the operation of the sterilization unit based on a signal transmitted from the detection unit.

In this case, the sterilization unit may be provided on the auxiliary unit to sterilize the first support surface.

In this case, one of the main body unit and the auxiliary unit may be provided with a guide rail extending in the longitudinal direction, and the other may be provided with a guide roller that moves along the guide rail, and the sterilization unit may be provided on an opposing surface arranged to face the second support surface.

In this case, a rear end of the main body unit in the longitudinal direction and a front end of the auxiliary unit in the longitudinal direction may be hinge-coupled to each other, and the sterilization unit may be provided on the second support surface.

In this case, side frames may be provided on both sides of the main body unit in a width direction, respectively, an extension surface extending to a position higher than the first support surface may be provided on the side frame, and the sterilization unit may be provided on the extension surface to sterilize the first support surface.

In this case, the first support surface may be disposed at a first height, and the sterilization unit may be disposed at a second height higher than the first height in the height direction.

In this case, when the massage module is not used, the second support surface may be disposed at a third height higher than the first height, and the third height may be lower than the second height.

In this case, the detection unit may include a first detection member configured to detect full closure in which the second support surface is disposed above the first support surface in the height direction, and the control unit may be configured to control the sterilization unit to operate when a full-closure signal is transmitted through the first detection member.

In this case, the control unit may be configured to determine first whether a thermo-massage operation has ended when a manual sterilization mode signal is transmitted from a user, and control the sterilization unit to operate when the full-closure signal is transmitted from the first detection member after the thermo-massage operation has ended.

In this case, the control unit may be configured to control the sterilization unit to operate when the full-closure signal is transmitted from the first detection member after a thermo-massage operation has ended, when an automatic sterilization mode signal is transmitted from a user.

In this case, the thermo-massage device may further include a detection unit provided with a second detection member configured to detect a position of a user, the control unit may be configured to control the sterilization unit to operate when a safety signal is transmitted through the second detection member.

In this case, the control unit may be configured to control operations of the sterilization unit such that the first support surface is first sterilized when the safety signal is transmitted through the second detection member, and provide a notification to a user that sterilization of the first support surface has been completed and preparation for sterilization of the second support surface is made.

In this case, the detection unit may further include a first detection member configured to detect full closure in which the second support surface is disposed above the first support surface in the height direction, and when the auxiliary unit is to be manually moved by a user, the control unit may be configured to control operations of the sterilization unit such that the second support surface is sterilized when the full-closure signal is transmitted through the first detection member and the safety signal is transmitted through the second detection member.

In this case, the detection unit may further include a first detection member configured to detect full closure in which the second support surface is disposed above the first support surface in the height direction, and the control unit may be configured to control operations of the sterilization unit such that the first support surface is first sterilized when the safety signal is transmitted through the second detection member, operate a sliding motor provided on the auxiliary unit to automatically move the auxiliary unit when sterilization of the first support surface is completed, and control operations of the sterilization unit such that the second support surface is sterilized when the full-closure signal is transmitted through the first detection member.

According to the above configuration, the thermo-massage device having a UV LED according to an embodiment of the present invention is configured to sterilize at least one of the first support surface and the second support surface through the sterilization unit equipped with a UV LED, thereby enabling effective sterilization even in a recumbent-type thermo-massage device used by a user while lying down.

In addition, a detection unit for detecting the presence of a user is provided, and based on a signal transmitted through the detection unit, the control unit controls the operation of the sterilization unit to prevent UV light from being directly irradiated onto the user. Particularly, based on the signal transmitted through the detection unit, if it is confirmed that the thermo-massage device is being stored, the control unit controls the sterilization unit such that at least one of the first support surface and the second support surface is sterilized, thereby preventing the user from suffering fatal injuries caused by UV light.

Furthermore, since the sterilization unit is provided on the auxiliary unit, when the thermo-massage device is used in a recumbent manner in which the user lies down, even if the user's weight is concentrated on the first support surface, the discomfort felt by the user can be minimized.

Advantageous effects of the present invention are not limited to the above-described effects, and should be understood to include all effects that can be inferred from the configuration of the invention described in the detailed description or claims of the present invention.

### BRIEF DESCRIPTION OF THE DRAWINGS

FIGS. 1 and 2 are cross-sectional views of a thermo-massage device having a UV LED according to an exemplary embodiment of the present invention, in which FIG. 1 shows the massage module in use, and FIG. 2 shows the massage module not in use.

FIGS. 3 and 4 are perspective views showing a portion of a thermo-massage device having a UV LED according to an exemplary embodiment of the present invention, in which FIG. 3 shows the auxiliary unit in a moving state, and FIG. 4 shows the auxiliary unit in a fully closed state.

FIGS. 5 and 6 are cross-sectional views of a thermo-massage device having a UV LED according to another exemplary embodiment of the present invention, in which FIG. 5 shows the massage module in use, and FIG. 6 shows the massage module not in use.

FIG. 7 is a front view of a thermo-massage device having a UV LED according to still another exemplary embodiment of the present invention.

FIGS. 8 and 9 are cross-sectional views of a thermo-massage device having a UV LED according to yet another exemplary embodiment of the present invention, in which FIG. 8 shows the massage module in use, and FIG. 9 shows the massage module not in use.

### DETAILED DESCRIPTION

Hereinafter, exemplary embodiments of the present invention will be described in detail so that those of ordinary skill in the art can readily implement the present invention with reference to the accompanying drawings. The present invention may be embodied in many different forms and is not limited to the embodiments set forth herein. In the drawings, parts unrelated to the description are omitted for clarity of description of the present invention, and throughout the specification, same or similar reference numerals denote same elements.

The words and terms used in the present specification and claims should not be interpreted as being limited to their ordinary or dictionary meanings, but should be construed as having meanings and concepts consistent with the technical spirit of the present invention, in accordance with the principle that an inventor may define terms and concepts to best describe their invention.

Accordingly, the embodiments described in the present specification and the configurations shown in the drawings correspond to preferred embodiments of the present invention, and do not represent all the technical spirit of the present invention, so the configurations may have various examples of equivalent and modification that can replace them at the time of filing the present invention.

It should be understood that the terms “comprise or include” or “have” or the like when used in this specification, are intended to describe the presence of stated features, numbers, steps, operations, elements, components and/or a combination thereof but not preclude the possibility of the presence or addition of one or more other features, numbers, steps, operations, elements, components, or a combination thereof.

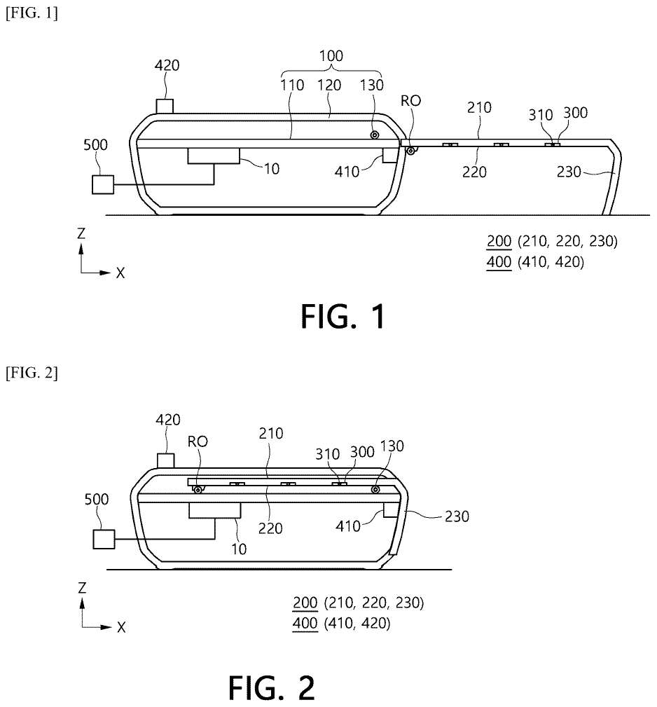

FIGS. 1 and 2 are cross-sectional views of a thermo-massage device having a UV LED according to an exemplary embodiment of the present invention, in which FIG. 1 shows the massage module in use, and FIG. 2 shows the massage module not in use. Here, the X direction is the longitudinal direction (the same direction as the arrangement direction of the user's spine), the Y direction is the width direction, and the Z direction is the height direction.

As shown in FIGS. 1 and 2, a thermo-massage device having a UV LED according to an exemplary embodiment of the present invention includes: a massage module 10 for thermo-massaging the body of a user; a main body unit 100 having a first support surface 110 for supporting a portion of the user's body; and an auxiliary unit 200 which has a second support surface 210 for supporting the remaining portion of the user's body, and which is movably provided so that, when the massage module 10 is in use, the first support surface 110 and the second support surface 210 are arranged in a line in the longitudinal direction (X), and when the massage module 10 is not in use, the second support surface 210 is arranged above the first support surface 110 in the height direction (Z).

Such a massage module 10 may be installed in the main body unit 100 for supporting a portion of the user's body, may be installed in the auxiliary unit 200 for supporting the remaining portion of the user's body, or may be installed in both the main body unit 100 and the auxiliary unit 200. The massage module 10 massages the user's body while moving in the longitudinal direction (X) in a state in which it is installed in the main body unit 100, the auxiliary unit 200, or both the main body unit 100 and the auxiliary unit 200.

For example, the massage module 10 may include a pressing part for pressing the user's body, a lifting/lowering part for raising or lowering the pressing part in the height direction (Z), and a transfer part for moving the massage module 10 in the longitudinal direction (X). However, the massage module 10 is not necessarily limited to such a configuration, and may also include a form that massages the user's body while moving only in the longitudinal direction (X) with a fixed position in the height direction (Z), or a form that massages the user's body while moving only in the height direction (Z) with a fixed position in the longitudinal direction (X).

In this case, the thermo-massage device having a UV LED according to an exemplary embodiment of the present invention includes a sterilization unit 300 having a UV LED 310 so as to sterilize at least one of the first support surface 110 and the second support surface 210, and a control unit 500 for controlling operations of the massage module and the sterilization unit 300.

That is, by being configured to sterilize at least one of the first support surface 110 and the second support surface 210 through the sterilization unit 300 having the UV LED 310, effective sterilization can also be achieved in a thermo-massage device of a recumbent-type used by a user in a lying posture.

The UV LED may irradiate ultraviolet rays of various wavelengths to at least one of the first support surface 110 and the second support surface 210, and, for example, may be configured to irradiate not only general UV light (typically in the UVC wavelength band) but also 172 nm excimer UV light in the vacuum ultraviolet wavelength range. In this case, an excimer lamp may be provided so as to irradiate vacuum ultraviolet rays. Vacuum ultraviolet (VUV) is ultraviolet light having a wavelength of 100 nm to 200 nm, which is close to X-rays among ultraviolet rays, and, due to its very high photon energy, enables effective sterilization.

In another example, the sterilization unit 300 may be configured to irradiate UV light using a discharge gas, and the discharge gas may be a discharge gas that generates excimer UV light such as Xe (wavelength: 172 nm), ArF (wavelength: 193 nm), KrCl (wavelength: 222 nm), KrF (wavelength: 248 nm), XeCl (wavelength: 308 nm), or XeF (wavelength: 351 nm), but a substance capable of generating ultraviolet rays in the UVC wavelength band (100 nm to 280 nm) may also be used.

In this case, the thermo-massage device may further include a detection unit 400 for detecting whether a user is present and whether the second support surface 210 is arranged above the first support surface 110 in the height direction (Z). That is, since the stronger the sterilizing power of the irradiated UV light, the more fatal it can be if it is directly irradiated to the user, it is important to configure the sterilization unit 300 to sterilize at least one of the first support surface 110 and the second support surface 210 only when the user is located at a safe distance or more. As described above, when the detection unit 400 for detecting the presence of a user is provided, the control unit 500 can control the operation of the sterilization unit 300 based on a signal transmitted from the detection unit 400 so as to prevent the UV light from being directly irradiated to the user.

In addition, the detection unit 400 also detects whether the second support surface 210 of the auxiliary unit 200 is arranged above the first support surface 110 of the main body unit 100 in the height direction (Z). When the massage module 10 is not in use, the auxiliary unit 200 is slid to be positioned above the main body unit 100 for storage. Based on a signal transmitted from the detection unit 400, if the control unit 500 determines that the thermo-massage device is in a stored state, the control unit 500 controls the sterilization unit 300 to sterilize at least one of the first support surface 110 and the second support surface 210, thereby preventing the user from suffering a fatal injury due to the UV light.

As shown in FIGS. 1 and 2, the sterilization unit 300 may be provided in the auxiliary unit 200 so as to sterilize the first support surface 110. That is, since odors or the like of the user may remain on the main body unit 100 as the thermo-massage is performed through the massage module 10 of the main body unit 100, it is important to configure the device so that the main body unit 100 can be intensively sterilized.

In this case, it is also important to ensure that the user does not feel discomfort due to the sterilization unit 300. For example, the main body unit 100 supports the upper body of the user, and the auxiliary unit 200 supports the lower body of the user. Generally, the upper body has a larger area and is heavier than the lower body, and since the thermo-massage device is used in a lying type in which the user lies down, the user's weight is concentrated on the first support surface 110. Therefore, as described above, when the sterilization unit 300 is provided in the auxiliary unit 200, discomfort that may be felt by the user due to the sterilization unit 300 can be minimized.

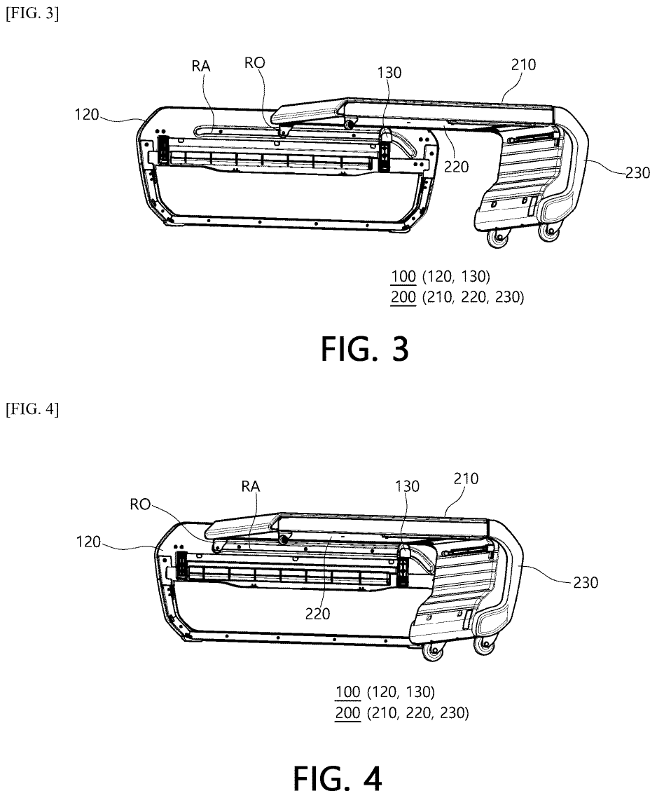

FIGS. 3 and 4 are perspective views showing a portion of a thermo-massage device having a UV LED according to an exemplary embodiment of the present invention, in which FIG. 3 shows the auxiliary unit in a moving state, and FIG. 4 shows the auxiliary unit in a fully closed state.

As shown in FIGS. 3 and 4, either the main body unit 100 or the auxiliary unit 200 is provided with a guide rail RA extending in the longitudinal direction (X), and the other is provided with a guide roller RO that moves along the guide rail RA, and the sterilization unit 300 may be provided on an opposing surface 220 that is arranged to face the second support surface 210.

For example, the guide rail RA may be provided on the inner surfaces of side frames 120 provided on both sides in the width direction (Y) of the main body unit 100, and may be horizontally arranged along the longitudinal direction (X). Such guide rails RA may be manufactured as a pair of rails as separate components and mounted to the inner surfaces of the side frames 120, or may be integrally formed with the side frames 120 during molding thereof, and, if necessary, may be configured to be attachable or detachable.

In addition, the guide roller RO described above may be configured in the form of a roller or a wheel so as to be slidable along the guide rail RA, and may be provided on both sides in the width direction (Y) of the auxiliary unit 200. In this case, the main body unit 100 may be provided with an auxiliary roller 130 for supporting the auxiliary unit 200. That is, during movement of the auxiliary unit 200, one side of the auxiliary unit 200 is supported by the guide roller RO, and the other side of the auxiliary unit 200 is supported by the auxiliary roller 130, so that the auxiliary unit 200 moves in a state where both sides are supported, thereby enabling the auxiliary unit 200 to move while maintaining a horizontal state. It is preferable that the auxiliary roller 130 and the guide roller RO be provided at a height at which the auxiliary unit 200 can be maintained in a horizontal state.

In another example, it is also possible to configure the guide roller RO described above to be provided in the main body unit 100 and the guide rail RA to be provided in the auxiliary unit 200.

As such, when the sterilization unit 300 is provided on the opposing surface 220 that is arranged to face the second support surface 210, it becomes possible to sterilize the first support surface 110 through the sterilization unit 300 while the auxiliary unit 200 is positioned above the main body unit 100 in the height direction (Z) when the massage module 10 is not in use. In addition, by providing the sterilization unit 300 on the opposing surface 220 of the auxiliary unit 200, discomfort that may be felt by the user due to the sterilization unit 300 can be minimized.

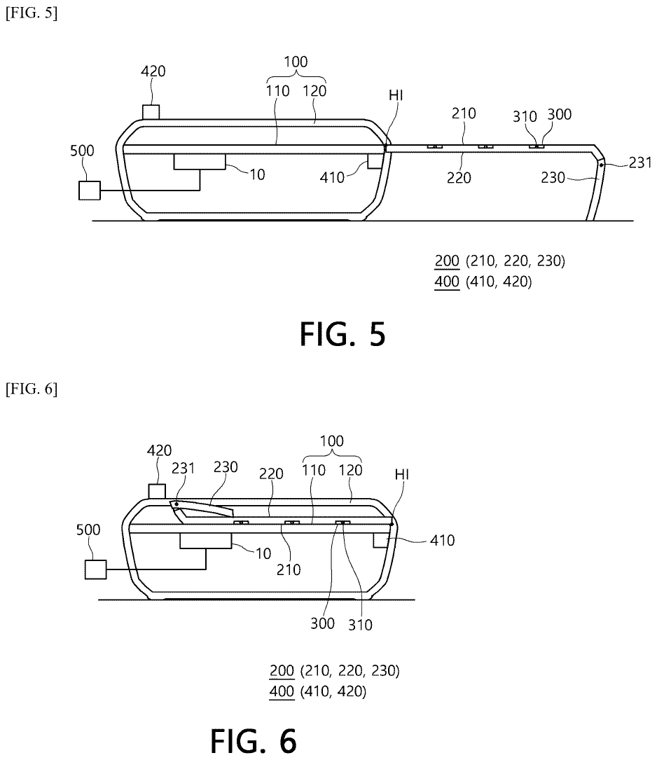

FIGS. 5 and 6 are cross-sectional views of a thermo-massage device having a UV LED according to another exemplary embodiment of the present invention, in which FIG. 5 shows the massage module in use, and FIG. 6 shows the massage module not in use.

As shown in FIGS. 5 and 6, the rear end in the longitudinal direction (X) of the main body unit 100 and the front end in the longitudinal direction (X) of the auxiliary unit 200 may be hinge-coupled to each other (HI). That is, the auxiliary unit 200 is configured to pivot in a hinge manner. In this case, the sterilization unit 300 may be provided on the second support surface 210. That is, when the massage module 10 is not in use, as the auxiliary unit 200 pivots, it becomes possible to sterilize the first support surface 110 through the sterilization unit 300 in a state in which the auxiliary unit 200 is positioned above the main body unit 100 in the height direction (Z). However, when the auxiliary unit 200 pivots in a hinge manner, if a leg 230 supporting the auxiliary unit 200 protrudes upward, there is a risk of safety accidents. Therefore, in a state in which the auxiliary unit 200 is pivoted, a leg hinge 231 may be provided so that the upwardly protruding leg 230 can be folded for storage.

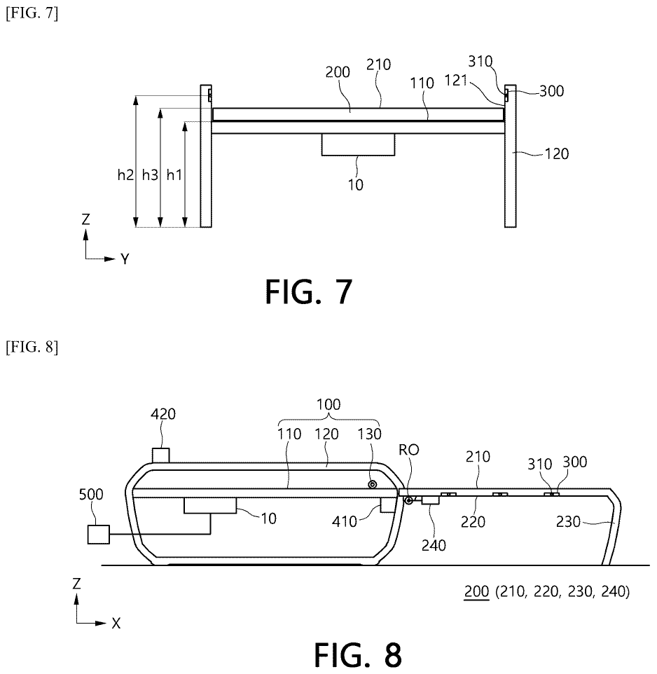

FIG. 7 is a front view of a thermo-massage device having a UV LED according to still another exemplary embodiment of the present invention.

As shown in FIG. 7, side frames 120 are respectively provided on both sides in the width direction (Y) of the main body unit 100, and the side frames 120 are provided with extension surfaces 121 extending to a position higher than the first support surface 110, and the sterilization unit 300 may be provided on the extension surfaces 121 so as to sterilize the first support surface 110. That is, the user may control the first support surface 110 to be sterilized by directly operating the sterilization unit 300 using a separate remote controller after a thermo-massage operation. In this case, since the configuration allows the user to directly operate the sterilization unit 300, the detection unit 400 for detecting the position of the user may not be necessary.

With this configuration, not only can the first support surface 110 be effectively sterilized, but by providing the sterilization unit 300 on the extension surfaces 121 of the side frames 120, discomfort that may be felt by the user due to the sterilization unit 300 can be minimized.

As shown in FIG. 7, the first support surface 110 is disposed at a first height h1, and the sterilization unit 300 may be disposed at a second height h2 that is higher than the first height h1 in the height direction (Z), thereby enabling effective sterilization of the first support surface 110.

As shown in FIG. 7, when the massage module 10 is not in use, the second support surface 210 is disposed at a third height h3 that is higher than the first height h1, and the third height h3 may be disposed lower than the second height h2. With this configuration, it is possible to sterilize not only the first support surface 110 but also the second support surface 210 through the sterilization unit 300.

That is, when the use of the massage module 10 is completed, the first support surface 110 is first sterilized, and after the sterilization of the first support surface 110 is completed, the auxiliary unit 200 is slid to be positioned above the main body unit 100, and then the second support surface 210 is sterilized. In this case, the user may control the first support surface 110 to be sterilized by directly operating the sterilization unit 300 using a separate remote controller after the thermo-massage operation, and after the sterilization of the first support surface 110 is completed, the user may directly slide the auxiliary unit 200 or use a sliding motor 240, which will be described later, to automatically slide the auxiliary unit 200, and then directly operate the sterilization unit 300 again to sterilize the second support surface 210. In this case, since the configuration allows the user to directly operate the sterilization unit 300, the detection unit 400 for detecting the position of the user may not be necessary.

As shown in FIGS. 1 and 2, the detection unit 400 may include a first detection member 410 for detecting a fully closed state in which the second support surface 210 is positioned above the first support surface 110 in the height direction (Z), and the control unit 500 may control the sterilization unit 300 to operate when a full-closure signal is transmitted through the first detection member 410.

That is, when a full-closure signal of the auxiliary unit 200 is transmitted through the first detection member 410, the control unit controls the sterilization unit 300 to start operating. In this case, when the sterilization unit 300 is configured to sterilize only the first support surface 110, a configuration for detecting the position of the user is not necessary. This is because the sterilization unit 300 operates after the fully closed state of the auxiliary unit 200 is detected, and when the auxiliary unit 200 is fully closed, the sterilization unit 300 is not exposed to the outside, thereby preventing the user from being injured by UV light.

As shown in FIGS. 1 and 2, when a manual sterilization mode signal is transmitted from the user, the control unit 500 may first determine whether the thermo-massage operation has been completed, and, after the thermo-massage operation is completed, may control the sterilization unit 300 to operate when a full-closure signal is transmitted from the first detection member 410.

That is, in the case where the user directly inputs the sterilization mode manually, the configuration allows manual input of the sterilization mode to perform sterilization while the thermo-massage device is stored. However, since the user may erroneously input the manual mode during the thermo-massage operation, in such a case, the control unit first determines whether the thermo-massage operation has been completed, and, when the thermo-massage operation is completed and the fully closed state of the auxiliary unit 200 is detected, controls the sterilization unit 300 to operate.

As shown in FIGS. 1 and 2, when an automatic sterilization mode signal is transmitted from the user, the control unit 500 may control the sterilization unit 300 to operate when a full-closure signal is transmitted from the first detection member 410 after the thermo-massage operation has been completed.

That is, in a case where the sterilization mode is automatically input according to the massage mode selected by the user, the control unit controls the sterilization unit 300 to operate when a full-closure signal is transmitted from the first detection member 410 after the thermo-massage operation has been completed.

As shown in FIGS. 1 and 2, the device includes a detection unit 400 provided with a second detection member 420 for detecting the position of the user, and the control unit 500 may control the sterilization unit 300 to operate when a safety signal is transmitted through the second detection member 420.

That is, the control unit 500 controls the sterilization unit 300 to operate when a safety signal is transmitted through the second detection member 420. In this case, the sterilization unit 300 can sterilize both the first support surface 110 and the second support surface 210, and since the sterilization unit 300 is exposed to the outside, it is important to configure the device so that the user is not injured by the sterilization unit 300. That is, the second detection member 420 transmits a safety signal only when the user is located at a distance equal to or greater than a safe distance at which the user cannot be harmed by the UV LED 310 of the sterilization unit 300. When the thermo-massage operation is completed and the user is at a safe distance or more, the sterilization unit 300 is controlled to operate to sterilize the first support surface 110.

As shown in FIGS. 1 and 2, when a safety signal is transmitted through the second detection member 420, the control unit 500 controls the operation of the sterilization unit 300 so that the first support surface 110 is sterilized first, and when the sterilization of the first support surface 110 is completed, the control unit 500 may provide a notification to the user indicating the completion of sterilization of the first support surface 110 and preparation for sterilization of the second support surface 210.

That is, since the sterilization unit 300 is provided on the side frames 120 of the main body unit 100, the first support surface 110 is sterilized first, and in order to sterilize the second support surface 210, the auxiliary unit 200 must be slid so as to be positioned above the main body unit 100 in the height direction (Z). In this case, when the configuration allows the user to manually move the auxiliary unit 200, the user confirms the notification of the completion of sterilization of the first support surface 110 and the preparation for sterilization of the second support surface 210, and then slides the auxiliary unit 200.

As shown in FIGS. 1 and 2, the detection unit 400 further includes a first detection member 410 for detecting a fully closed state in which the second support surface 210 is positioned above the first support surface 110 in the height direction (Z). When the user is required to manually move the auxiliary unit 200, the control unit 500 may control the sterilization unit 300 to sterilize the second support surface 210 when a full-closure signal is transmitted through the first detection member 410 and a safety signal is transmitted through the second detection member 420.

That is, since the user needs to approach again after sterilization of the first support surface 110 to slide the auxiliary unit 200, it is necessary to confirm that the user is at a safe distance or more before operating the sterilization unit 300 for sterilization of the second support surface 210.

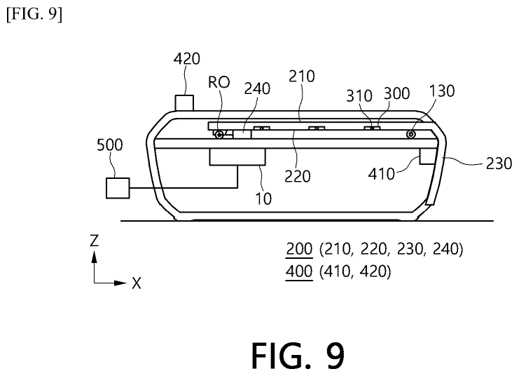

FIGS. 8 and 9 are cross-sectional views of a thermo-massage device having a UV LED according to yet another exemplary embodiment of the present invention, in which FIG. 8 shows the massage module in use, and FIG. 9 shows the massage module not in use.

As shown in FIGS. 8 and 9, the detection unit 400 further includes a first detection member 410 for detecting a fully closed state in which the second support surface 210 is positioned above the first support surface 110 in the height direction (Z). When a safety signal is transmitted through the second detection member 420, the control unit 500 controls the operation of the sterilization unit 300 so that the first support surface 110 is sterilized first. When the sterilization of the first support surface 110 is completed, the control unit operates the sliding motor 240 provided in the auxiliary unit 200 so that the auxiliary unit 200 is automatically moved, and when a full-closure signal is transmitted through the first detection member 410, the control unit controls the operation of the sterilization unit 300 so that the second support surface 210 is sterilized.

That is, when the auxiliary unit 200 is configured to be automatically slidable through the sliding motor 240, the auxiliary unit 200 is automatically moved after the sterilization of the first support surface 110, so the user does not need to approach again. Accordingly, it is configured such that, after the sterilization of the first support surface 110, the auxiliary unit 200 is automatically moved, and once the full-closure signal of the auxiliary unit 200 is transmitted, the sterilization of the second support surface 210 is carried out.

With this configuration, not only can the user be protected, but sterilization of the first support surface 110 and the second support surface 210 can also be achieved in a simple manner.

As described above, the thermo-massage device having a UV LED according to an exemplary embodiment of the present invention is configured to sterilize at least one of the first support surface 110 and the second support surface 210 through the sterilization unit 300 having the UV LED 310, thereby enabling effective sterilization even for a recumbent-type thermo-massage device used by a user in a lying posture. In addition, the detection unit 400 for detecting the presence or absence of a user is provided, and the control unit 500 controls the operation of the sterilization unit 300 based on a signal transmitted from the detection unit 400 to prevent UV light from being directly irradiated to the user. In particular, when it is determined, based on a signal transmitted from the detection unit 400, that the thermo-massage device is in storage, the control unit 500 controls the sterilization unit 300 so that at least one of the first support surface 110 and the second support surface 210 is sterilized, thereby preventing the user from suffering a fatal injury due to the UV light. Furthermore, since the sterilization unit 300 is provided in the auxiliary unit 200, even when the thermo-massage device is used in a lying type in which the user lies down and the user's weight is concentrated on the first support surface 110, discomfort that may be felt by the user can be minimized.

Although exemplary embodiments of the present invention have been described above, the spirit of the present invention is not limited to the embodiments set forth herein. Those of ordinary skill in the art who understand the spirit of the present invention may easily propose other embodiments through supplement, change, removal, addition, etc. of elements within the same spirit, but the embodiments will be also within the scope of the present invention.

1. A thermo-massage device having a UV LED, comprising:
a main body unit having a massage module configured to thermo-massage a body of a user and a first support surface for supporting a portion of the body of the user;
an auxiliary unit which has a second support surface for supporting a remaining portion of the body of the user, and which is movably provided so that, when the massage module is in use, the first support surface and the second support surface are arranged in a line in a longitudinal direction, and, when the massage module is not in use, the second support surface is arranged above the first support surface in a height direction;
a sterilization unit having a UV LED configured to sterilize at least one of the first support surface and the second support surface; and
a control unit configured to control operations of the massage module and the sterilization unit.

2. The thermo-massage device having a UV LED according to claim 1,
further comprising a detection unit configured to detect whether a user is present and whether the second support surface is arranged above the first support surface in the height direction,
wherein the control unit is configured to control the operation of the sterilization unit based on a signal transmitted from the detection unit.

3. The thermo-massage device having a UV LED according to claim 2, wherein the sterilization unit is provided on the auxiliary unit to sterilize the first support surface.

4. The thermo-massage device having a UV LED according to claim 3,
wherein one of the main body unit and the auxiliary unit is provided with a guide rail extending in the longitudinal direction, and the other is provided with a guide roller that moves along the guide rail,
and the sterilization unit is provided on an opposing surface arranged to face the second support surface.

5. The thermo-massage device having a UV LED according to claim 3,
wherein a rear end of the main body unit in the longitudinal direction and a front end of the auxiliary unit in the longitudinal direction are hinge-coupled to each other, and the sterilization unit is provided on the second support surface.

6. The thermo-massage device having a UV LED according to claim 1,
wherein side frames are provided on both sides of the main body unit in a width direction, respectively,
an extension surface extending to a position higher than the first support surface is provided on the side frame, and
the sterilization unit is provided on the extension surface to sterilize the first support surface.

7. The thermo-massage device having a UV LED according to claim 6,
wherein the first support surface is disposed at a first height,
and the sterilization unit is disposed at a second height higher than the first height in the height direction.

8. The thermo-massage device having a UV LED according to claim 7, wherein, when the massage module is not used, the second support surface is disposed at a third height higher than the first height, and the third height is lower than the second height.

9. The thermo-massage device having a UV LED according to claim 3,
wherein the detection unit comprises a first detection member configured to detect full closure in which the second support surface is disposed above the first support surface in the height direction,
and the control unit is configured to control the sterilization unit to operate when a full-closure signal is transmitted through the first detection member.

10. The thermo-massage device having a UV LED according to claim 9, wherein the control unit is configured to determine first whether a thermo-massage operation has ended when a manual sterilization mode signal is transmitted from a user, and control the sterilization unit to operate when the full-closure signal is transmitted from the first detection member after the thermo-massage operation has ended.

11. The thermo-massage device having a UV LED according to claim 9, wherein the control unit is configured to control the sterilization unit to operate when the full-closure signal is transmitted from the first detection member after a thermo-massage operation has ended, when an automatic sterilization mode signal is transmitted from a user.

12. The thermo-massage device having a UV LED according to claim 6,
further comprising a detection unit provided with a second detection member configured to detect a position of a user,
wherein the control unit is configured to control the sterilization unit to operate when a safety signal is transmitted through the second detection member.

13. The thermo-massage device having a UV LED according to claim 12,
wherein the control unit is configured to:
control operations of the sterilization unit such that the first support surface is first sterilized when the safety signal is transmitted through the second detection member, and
provide a notification to a user that sterilization of the first support surface has been completed and preparation for sterilization of the second support surface is made.

14. The thermo-massage device having a UV LED according to claim 13,
wherein the detection unit further comprises a first detection member configured to detect full closure in which the second support surface is disposed above the first support surface in the height direction,
and when the auxiliary unit is to be manually moved by a user, the control unit is configured to control operations of the sterilization unit such that the second support surface is sterilized when the full-closure signal is transmitted through the first detection member and the safety signal is transmitted through the second detection member.

15. The thermo-massage device having a UV LED according to claim 12,
wherein the detection unit further comprises a first detection member configured to detect full closure in which the second support surface is disposed above the first support surface in the height direction, and
wherein the control unit is configured to:
control operations of the sterilization unit such that the first support surface is first sterilized when the safety signal is transmitted through the second detection member,
operate a sliding motor provided on the auxiliary unit to automatically move the auxiliary unit when sterilization of the first support surface is completed, and
control operations of the sterilization unit such that the second support surface is sterilized when the full-closure signal is transmitted through the first detection member.
